(19) United States
(12) Patent Application Publication
(10) Pub. No.: US 2007/0123507 A1
(43) Pub. Date: May 31, 2007

(54) Process for converting heterocyclic ketones to amido-substituted heterocycles

(75) Inventor: Thomas A. Brandt, NORTH STONINGTON, CT (US)

Correspondence Address: PFIZER INC., PATENT DEPARTMENT, MS8260-1611, EASTERN POINT ROAD, GROTON, CT, 06340, US

(73) Assignee: PFIZER INC.

(21) Appl. No.: 10/582,681

(22) PCT Filed: Nov. 22, 2004
(86) PCT No.: PCT/IB04/03815

§ 371 Date: Jun. 13, 2006

Related U.S. Application Data
(60) Provisional application No. 60/526,546, filed on Dec. 2, 2003.

(51) Int. Cl. A61K 31/55; A61K 31/445; A61K 31/4747; C07D 471/10

(52) U.S. Cl. 514/212.02; 514/217.11; 514/278; 514/329; 540/543; 540/605; 546/16; 546/244

(57) ABSTRACT

The present invention provides a safe and convenient process for preparing compounds of Formula (I) from the corresponding heterocyclic ketone.

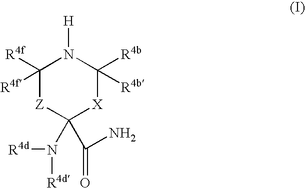

The present invention relates to an improved process for preparing amido-substituted 4- to 6-membered heterocyclic compounds from 4- to 6-membered heterocyclic ketones. The amido-substituted 4- to 6-membered heterocyclic compounds are useful intermediates in the synthesis of cannabinoid (CB-1) antagonists.

### BACKGROUND

The synthesis of α-amino acids by reaction of aldehydes with ammonia and hydrogen cyanide followed by hydrolysis of the resulting α-aminonitriles is known as the Strecker Amino-Acid Synthesis. See, A. Strecker, Ann, 75, 27 (1850); and A. Strecker, Ann, 91, 349 (1854). Over the years, safer, milder, and more selective reaction conditions have been developed, especially in regard to asymmetric synthesis. In addition, the scope of the reaction has been extended to include primary and secondary amines. See, e.g., J. P. Greenstein, M. Winitz, Chemistry of the Amino Acids, vol. 3 (New York, 1961) pp 698-700; G. C. Barrett, “Asymmetric synthesis using enantiopure sulfinimines”, Chemistry and Biochemistry of the Amino Acids (Chapman and Hall, New York, 1985) pp 251, 261.; F. A. Davis, et al., “Review of Stereoselective Synthesis”, Tetrahedron Letters, 35, 9351 (1994); R. O. Duthaler, Tetrahedron, 50, 1539-1650 passim (1994).

Although the Strecker reaction provides a convenient means for making α-aminonitriles, the use of cyanide reagents raises safety issues due to the high toxicity of any residual cyanide in the reaction mixture. Therefore, there is a need for an efficient means for producing an α-aminoamide from the corresponding α-aminonitrile without the risk of exposure to residual cyanide from the preparation of the intermediate α-aminonitrile.

### SUMMARY

The present invention provides a process for preparing a compound of Formula (I) having little or no risk of exposure to residual cyanide. 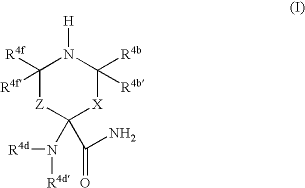
wherein

R^{4b} and R^{4b′} are each independently hydrogen or (C₁-C₆)alkyl;

X is a bond, —CH₂CH₂— or —C(R^{4c})(R^{4c′})—, where R^{4c} and R^{4c′} are each independently hydrogen or (C₁-C₆)alkyl;

R^{4d} is hydrogen, (C₁-C₆)alkyl, (C₃-C₆)cycloalkyl, or taken together with R^{4d′} forms a 4- to 6-membered heterocyclic ring optionally containing an additional heteroatom selected atom N, O, or S;

R^{4d′} is hydrogen, (C₁-C₆)alkyl, or taken together with R^{4d} forms a 4- to 6-membered heterocyclic ring optionally containing an additional heteroatom selected from N, O, or S;

Z is a bond, —CH₂CH₂−, or —C(R^{4e})(R^{4e′})—, where R^{4e} and R^{4e′} are each independently hydrogen or (C₁-C₆)alkyl; and

R^{4f} and R^{4f′} are each independently hydrogen or (C₁-C₆)alkyl;

or a pharmaceutically acceptable salt thereof; comprising the steps of

(1) reacting a compound having a, formula R^{4d}—NH—R^{4d′} and a cyanide source with a compound of Formula (Ia) to form an intermediate of Formula (Ib) 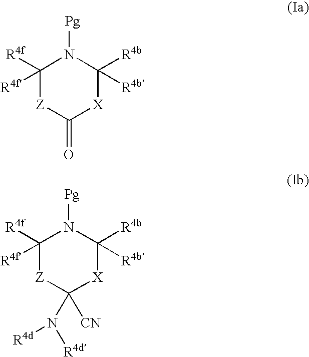
where Pg is a amino-protecting group and R^{4b}, R^{4b′}, X, Z, R^{4d}, R^{4′}, R^{4f} and R^{4f′} are as defined above;

(2) hydrolyzing the nitrile group of the compound of Formula (Ib) with alkaline hydrogen peroxide in the presence of dimethylsulfoxide to form a compound of Formula (Ic) 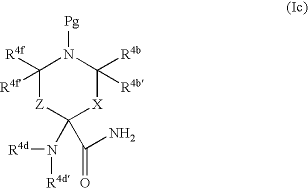
where Pg, R^{4b}, R^{4b′}, X, Z, R^{4d}, R^{4d}, R^{4d′}, R^{4f} and R^{4f′} are as defined above;

(3) removing the amino-protecting group to form the compound of Formula (I); and

(4) optionally forming a pharmaceutically acceptable salt of said compound of Formula (I).

Preferably, the compound of Formula (Ia) is converted to the compound of Formula (Ic) without isolating the compound of Formula (Ib).

For the compounds of Formula (I) and corresponding intermediates, R^{4b}, R^{4b′}, R^{4f}, R^{4f′} are preferably all hydrogens. X is preferably —CH₂— or a bond. Z is preferably —CH₂— or a bond (more preferably, X and Z are both a bond). R^{4d} is preferably (C₁-C₆)alkyl (more preferably, R^{4d} is ethyl) and R^{4d′} is preferably.hydrogen.

### DEFINITIONS

As used herein, the term “alkyl” refers to a hydrocarbon radical of the general formula CₙH₂ₙ₊₁. The alkane radical may be straight or branched. For example, the term “(C₁-C₆)alkyl” refers to a monovalent, straight, or branched aliphatic group containing 1 to 6 carbon atoms (e.g., methyl, ethyl, n-propyl, i-propyl, n-butyl, i-butyl, s-butyl, t-butyl, n-pentyl, 1-methylbutyl, 2-methylbutyl, 3-methylbutyl, neopentyl, 3,3-dimethylpropyl, hexyl, 2-methylpentyl, and the like). Similarly, the alkyl portion (i.e., alkyl moiety) of an alkylamino group has the same definition as above. The term “di(C₁-C₆)alkyl” refers to two (C₁-C₆)alkyl groups which may be the same or different.

The term “cycloalkyl” refers to a carbocyclic ring system which may include alkyl substitutions. For example, (C₃-C₆)cycloalkyl includes cyclopropyl, methylcyclopropyl, cyclobutyl, methylcyclobutyl, dimethylcyclobutyl, cyclopentyl, methylcyclopentyl, and cyclohexyl.

The term “cyanide source” refers to any reagent that can provide a cyanide ion under the reaction conditions. For example, potassium cyanide, sodium cyanide, trimethylsilyl cyanide, hydrogen cyanide, and the like.

The phrase “pharmaceutically acceptable” indicates that the substance or composition must be compatible chemically and/or toxicologically, with the other ingredients.

The term “protecting group” or “Pg” refers to a substituent that is commonly employed to block or protect a particular functionality while reacting other functional groups on the compound. For example, an “amino-protecting group” is a substituent attached to an amino group that blocks or protects the amino functionality in the compound. A preferred amino-protecting is benzhydryl.

### DETAILED DESCRIPTION

The process of the present invention provides a convenient and efficient means for preparing intermediates that are useful in making compounds that have been found to be cannabinoid (CB-1) antagonists. The starting materials for the process described herein are generally available from commercial sources such as Aldrich Chemicals (Milwaukee, Wis.) or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)).

Scheme I below illustrates the general process of the present invention.

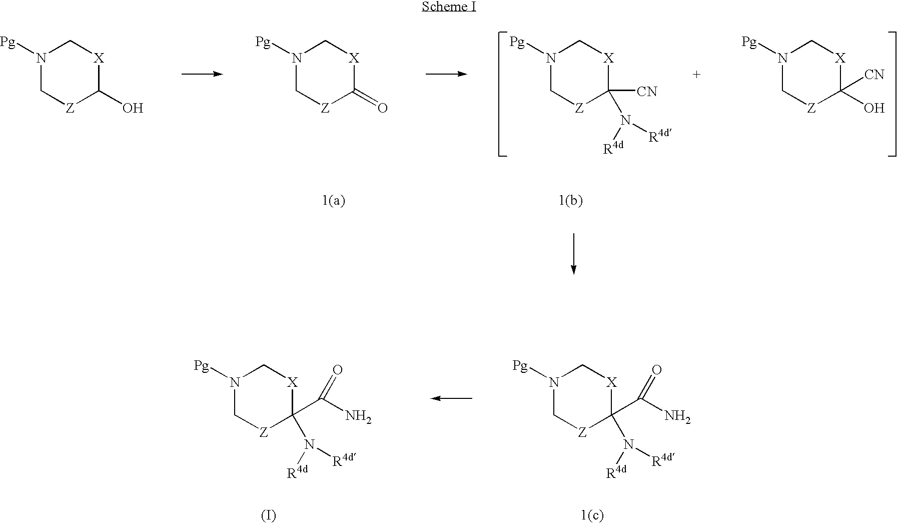

The amino group of the starting hydroxy compound is first protected prior to oxidation to the ketone intermediate 1(a) Alternatively when benzyhydryl is desired as the protecting group, the protected amino alcohol may be prepared directly by reacting benzhydryl amine with epichlorohydrin. Other amino-protecting groups may be used so long as the protecting group remains intact through out the process illustrated above. For example, it does not cleave under the acidic alcohol conditions of the Strecker reaction used to form the nitrilel (b) and does not cleave under the basic aqueous conditions during the hydrolysis of the nitrilel (b) to form the amide 1(d). The hydroxy group of the amino-protected starting material may be oxidized to the ketone using conventional oxidation procedures. For example, the hydroxy compound may be treated with oxalyl chloride and dimethyl sulfoxide in the presence of a base (e.g., triethylamine) to form the ketone 1(a) (also known as the Swern oxidation). The ketone 1(a) is reacted with the desired amino compound (R^{4d}—NH R^{4d′}, where R^{4d} and R^{4d′} are as defined above) and a cyanide source in a protic solvent (e.g., methanol and/or water) to form the nitrile 1(b). Suitable amino compounds include alkylamines (e.g., methyl amine, ethyl amine, n-proprylamine, iso-propyl amine, n-butylamine, sec-butylamine, iso-butyl amine, and the like.), dialkylamines (e.g., dimethylamine, diethylamine, methylethylamine, and the like), cycloalkylamines (e.g., cyclopropylamine, methylcyclopropylamine, cyclobutylamine, methylcyclobutylamine, dimethylcyclobutylamine, cyclopentylamine, methylcyclopentylamine, cyclohexylamine, and the like), and heterocyclic amines (e.g., azetidine, pyrrolidine, imidazolidine, oxazolidine, thiazolidine, piperidine, piperazine, morpholine, thiamorpholine, and the like). When a cyanide salt is used for the cyanide source, then the reaction medium needs to be acidic for the generation of hydrogen cyanide. For example, acetic acid or hydrochloric acid is typically added with potassium cyanide. The nitrile intermediate 1(b) is then hydrolyzed to the amide 1(c) using procedures analogous to those described by Yasuhiko Sawaki and Yoshiro Ogata in Bull Chem Soc Jpn, 54, 793-799 (1981). For example, nitrile intermediate 1(b) is treated with about 1.1 equivalents of alkaline hydrogen peroxide (e.g., hydrogen peroxide in the presence of a strong base (e.g., sodium hydroxide or potassium hydroxide) in the presence of about 1.2 equivalents of dimethylsulfoxide (DMSO) in a protic solvent (e.g., methanol). Generally, the amount of sodium hydroxide added is about 3 mol % over the amount of total acid used in the Strecker reaction (e.g., mol acetic acid plus mol HCI from amine hydrochloride salt) The pH is about 13. Preferably, the hydrolysis to the amide 1(c) is performed with the crude reaction mixture from the previous step without isolating the α-aminonitrile intermediate 1(b). Finally, the protecting group may be removed using procedures appropriate for the particular protecting group utilized. For example, when benzhydryl is the protecting group, it may be removed by hydrogenation in the presence of a catalyst (e.g., Pd(OH)₂).

There are several advantages of the process of the present invention over other processes that could be used for this conversion. For example, the introduction of the nitrile group into the molecule and the subsequent hydrolysis to the amide can be done in a single pot reaction. When X and Z are both a bond and R^{4d} is ethylamino, the amide 1(c) was isolated directly from the crude reaction mixture in sufficient purity to be used in the next step without any further purification, thus providing an efficiency advantage in manufacturing. In addition, the oxidizing agent (basic hydrogen peroxide) likely decomposes any remaining cyanide, presumably to cyanate and then further to carbon dioxide and ammonia, thus eliminating the safety issue associated with cyanide exposure and waste stream management. The use of basic peroxide hydrolysis allowed the reaction to take place in the presence of amine functionality which under neutral or slightly acidic H₂O₂— would likely have oxidized the tertiary amine to an N-oxide and the secondary amine to an oxime. In the present invention, the rate of nitrile hydrolysis is essentially instantaneous such that oxidative side reactions are relatively slow if present at all.

### EXAMPLES

Unless specified otherwise, starting materials are generally available from commercial sources such as Aldrich Chemicals Co. (Milwaukee, Wis.), Lancaster Synthesis, Inc. (Windham, N.H.), Acros Organics (Fairlawn, N.J.), Maybridge Chemical Company, Ltd. (Cornwall, England), Tyger Scientific (Princeton, N.J.), and AstraZeneca Pharmaceuticals (London, England).

### General Experimental Procedures

NMR spectra were recorded on a Varian Unity™ 400 or 500 (available from Varian Inc., Palo Alto, Calif.) at room temperature at 400 and 500 MHz ¹H, respectively. Chemical shifts are expressed in parts per million (6) relative to residual solvent as an internal reference. The peak shapes are denoted as follows: s, singlet; d, doublet; t, triplet; q, quartet; m, multiplet; br s, broad singlet; v br s, very broad singlet; br m, broad multiplet; 2s, two singlets. In some cases only representative ¹H NMR peaks are given.

Mass spectra were recorded by direct flow analysis using positive and negative atmospheric pressure chemical ionization (APcI) scan modes. A Waters APcl/MS model ZMD mass spectrometer equipped with Gilson 215 liquid handling system was used to carry out the experiments.

Mass spectrometry analysis was also obtained by RP-HPLC gradient method for chromatographic separation. Molecular weight identification was recorded by positive and negative electrospray ionization (ESI) scan modes. A Waters/Micromass ESI/MS model ZMD or LCZ mass spectrometer equipped with Gilson 215 liquid handling system and HP 1100 DAD was used to carry out the experiments. 1-Benzhydryl-azetidin-3-ol is available from DCI Pharmtech, Inc. (Taiwan)

### Example 1

### Preparation of 1-Benzhydryl-azetidin-3-one (I-1a)

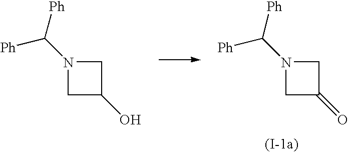

Oxalyl chloride (145.2 g, 1.121 mol) was added to dichloromethane (3.75 liters) and the resulting solution was cooled to −78° C. Methyl sulfoxide (179.1 g, 2.269 mol) was then added over a duration of 20 minutes (maintained internal temperature <−70° C. during addition). 1-Benzhydryl-azetidin-3-ol (250.0 g, 1.045 mol) was then added as a solution in dichloromethane (1.25 liter) to −78° C. solution over a duration of 40 minutes (maintained internal temperature <−70° C. during addition). The solution was stirred for 1 hour at −78° C. followed by the addition of triethylamine (427.1 g, 4.179 mol) over 30 minutes (maintained internal temperature <−70° C. during addition). Reaction was then allowed to come to room temperature slowly and stir for 20 hours. 1.0 M hydrochloric acid (3.2 liters, 3.2 mol) was added to the crude reaction solution over 30 minutes, followed by stirring for 10 minutes at room temperature. The heavy dichloromethane layer (clear yellow in color) was then separated and discarded. The remaining acidic aqueous phase (clear, colorless) was treated with 50% sodium hydroxide (150 ml, 2.1 mol) with stirring over a 30 minute period. The final aqueous solution had a pH=9. At this pH, the desired product precipitates from solution as a white solid. The pH=9 solution was stirred for 30 minutes and then the precipitated product was collected by filtration. The collected solid was washed with 1.0 liter of water and then air dried for 36 hr to give 1-benzhydryl-azetidin-3-one (I-1a) (184.1 g, 74%) as an off-white solid.

+ESI MS (M+1) 256.3 (M+1 of hydrated ketone); ¹H NMR (400 MHz, CD₂Cl₂)δ 7.47-7.49 (m, 4H), 7.27-7.30 (m, 4H), 7.18-7.22 (m, 2H), 4.60 (s, 1H), 3.97 (s, 4H).

### Preparation of 1-Benzhydryl-3-ethylamino-azetidine-3-carboxylic acid amide (I-1c)

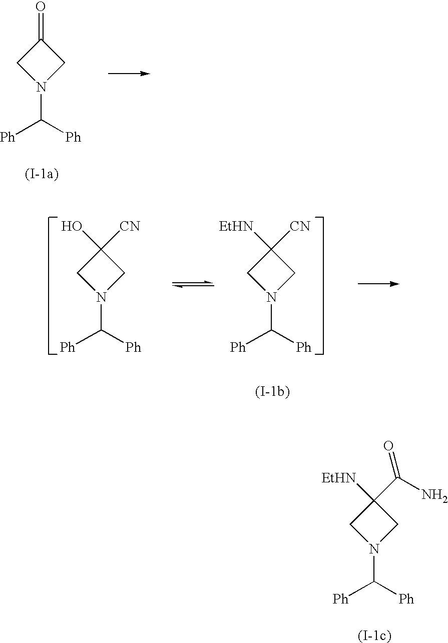

1-Benzhydryl-azetidin-3-one I-1a (53.43 g, 0.225 mol) was dissolved in methanol (750 ml) to give a clear pale yellow solution. Ethylamine hydrochloride (20.23 g, 0.243 mol) was added in one portion as a solid (reaction solution remains clear) followed by addition of potassium cyanide (15.38 g, 0.229 mol) in one portion as a solid (potassium cyanide not very methanol soluble—suspended as white flakes). Acetic acid (14.86 g, 0.246 mol) was added followed by stirring for 2.5 hours at room temperature to give a homogenous suspension (white crystalline solids of uniform small size). LCMS showed nearly complete consumption of azetidinone starting material and a mixture of 1-benzhydryl-3-hydroxy-azetidine-3-carbonitrile (cyanohydrin) and 1-benzhydryl-3-ethylamino-azetidine-3-carbonitrile (Strecker product). The reaction mixture was then warmed to 55° C. and stirred for 15 hours and LCMS analysis showed a ˜90:10 mixture of Strecker product:cyanohydrin (ratio appears to be an equilibrium ratio).

The crude reaction mixture was cooled to 50° C. followed by the addition of dimethyl sulfoxide (21.10 g, 0.269 mol) and then addition of aqueous 2N sodium hydroxide (251 ml, 0.502 mol) over 10 minutes (maintained internal temperature >45° C.). Re-analysis by LCMS shows all of the cyanohydrin was converted back to 1-benzhydryl-azetidin-3-one starting material to show a ratio of Strecker product:azetidinone of ˜90:10). Solution pH equaled13. To the basic reaction solution at 50° C. was added 11% aqueous hydrogen peroxide (80 ml, 0.247 mol) over 5 minutes while maintaining the internal temperature between 50 to 65° C. The product began to precipitate during peroxide addition, and after complete addition, water was added (270 ml) to help facilitate stirring. The reaction mixture was held at 50° C. for 30 minutes then cooled to room temperature over 1 hour, followed by stirring at room temperature for 1 hour. The precipitated product was collected by filtration and rinsed with 1.0 liter of water, followed by briefly air-drying on the filter for 1 hour. After further drying in vacuo, 1-benzhydryl-3-ethylamino-azetidine-3-carboxylic acid amide (I-1c) was isolated as an off-white solid (55.31 g, 79% over two steps).

+ESI MS (M+1) 310.5; ¹H NMR (400 MHz, CD₃OD) δ 7.41 (d, J=7.1 Hz, 4H), 7.25 (t, J=7.5 Hz, 4H), 7.16 (t, J=7.5 Hz, 2H), 4.49 (s,1 H) 3.44 (d, J=8.3 Hz, 2H), 3.11 (d, J=8.3 Hz, 2H), 2.47 (q, J=7.1 Hz, 2H), 1.10 (t, J=7.3 Hz, 3H).

### Preparation of 3-Ethylamino azetidine-3-carboxylic Acid Amide, Hydrochloride Salt (I)

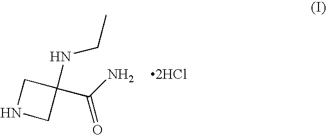

To a suspension of 1-benzhydryl-3-ethylaminoazetidine-3-carboxylic acid amide (I-1c; 36.1 g, 117 mmol) in methanol (560 ml) at room temperature was added concentrated aqueous HCI (19.5 ml, 234 mmol), resulting in a clear solution. To 20% Pd(OH)₂ on carbon (3.75 g) was added methanol (85 ml), followed by the methanolic solution of I-1c. The mixture was placed on a Parr® shaker and then reduced (50 psi H₂) at room temperature for 20 hours. The reaction was then filtered through Celite® and then concentrated to low volume under reduced pressure, at which point a precipitate forms. The suspension was diluted with 500 ml of methyl t-butyl ether (MTBE), stirred for an additional hour, and the precipitate collected by vacuum filtration. The solid was washed with MTBE and then dried, in vacuo, to afford (I) (24.8 g, 98%) as a colorless solid.

+APcI MS (M+1) 144.1; ¹H NMR (400 MHz, CD₂Cl₂) δ 4.56 (br s, 4H), 3.00 (q, J=7.2 Hz, 2H), 1.36 (t, J=7.1 Hz, 3H).

What is claimed is:

1. A process for preparing a compound of Formula (I) 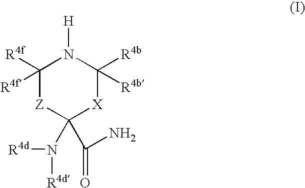
wherein
R^{4b} and R^{4b′} are each independently hydrogen or (C₁-C₆)alkyl;
X is a bond, —CH₂CH₂— or —C(R^{4c})(R^{4c′})—, where R^{4c} and R^{4c′} are each independently hydrogen or (C₁-C₆)alkyl;
R^{4d} is hydrogen, (C₁-C₆)alkyl, (C₃-C₆)cycloalkyl, or taken together with R^{4d′} forms a 4- to 6-membered heterocyclic ring optionally containing an additional heteroatom selected atom N, O, or S;
R^{4d′} is hydrogen, (C₁-C₆)alkyl, or taken together with R^{4d} forms a 4- to 6-membered heterocyclic ring optionally containing an additional heteroatom selected from N, O, or S;
Z is a bond, —CH₂CH₂—, or —C(R^{4e})(R^{4e′})—, where R^{4e} and R^{4e′} are each independently hydrogen or (C₁-C₆)alkyl; and
R^{4f} and R^{4f′} are each independently hydrogen or (C₁-C₆)alkyl;
or a pharmaceutically acceptable salt thereof;
comprising the steps of
(1) reacting a compound having a formula R^{4d}—NH—R^{4d′} and a cyanide source with a compound of Formula (Ia) to form an intermediate of Formula (Ib) 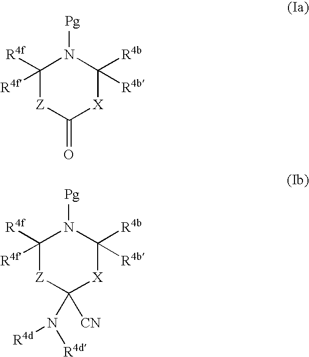
where Pg is a amino-protecting group and R^{4b}, R^{4b′}, X, Z, R^{4d}, R^{4d′}, R^{4f} and R^{4f′} are as defined above;
(2) hydrolyzing the nitrile group of the compound of Formula (Ib) with alkaline hydrogen peroxide in the presence of dimethylsulfoxide to form a compound of Formula (Ic) 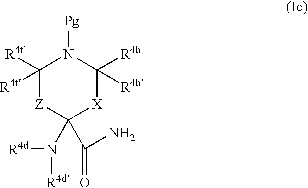
where Pg, R^{4b}, R^{4b′}, X, Z, R^{4d}, R^{4d}, R^{4d′}, R^{4f} and R^{4f′} are as defined above;
(3) removing the amino-protecting group to form the compound of Formula (I); and
(4) optionally forming a pharmaceutically acceptable salt of said compound of Formula (I).

2. The process of claim 1 wherein said compound of Formula (Ia) is converted to said compound of Formula (Ic) without isolating said compound of Formula (Ib).

3. The process of claim 2 wherein R^{4b}, R^{4b′}, R^{4f}, R^{4f′} are all hydrogens.

4. The process of claim 3 wherein X is —CH₂— or a bond; and Z is —CH₂— or a bond.

5. The process of claim 4 wherein R^{4d} is (C₁-C₆)alkyl and R^{4d′} is hydrogen.

6. The process of claim 5 wherein X and Z are both a bond.

7. The process of claim 5 or 6 wherein R^{4d} is ethyl.
